(19) United States
(12) Patent Application Publication
(10) Pub. No.: US 2026/0227353 A1
(43) Pub. Date: Aug. 6, 2026

(54) SYSTEM FOR TESTING THE VIABILITY FOR GENE BANK CONSERVATION OF PLANT GENETIC RESOURCES AND/OR FOR THE IDENTIFICATION AND SEPARATION OF VARIETIES/GROUPS OF VARIETIES, THE RELATED METHOD AND USE THEREOF

(71) Applicant: NEMZETI BIODIVERZITÁS ÉS GÉNMEGORZÉSI KÖZPONT, Tápiószele (HU)

(72) Inventors: Borbála BAKTAY, Badacsonytomaj (HU); József BERKE, Keszthely (HU); Ferenc GYULAI, Szarvasgede (HU); Tamás NAGY, Budapest (HU); Erno Ottó SZALKOVSZKI, Sárospatak (HU); Veronika KOZMA-BOGNÁR, Keszthely (HU)

(73) Assignee: NEMZETI BIODIVERZITÁS ÉS GÉNMEGORZÉSI KÖZPONT, Tápiószele (HU)

(21) Appl. No.: 19/152,332

(22) PCT Filed: Jan. 29, 2024
(86) PCT No.: PCT/HU2024/050005

§ 371 Date: Jul. 30, 2025

(30) Foreign Application Priority Data
Jan. 30, 2023 (HU) P2300042

(51) Int. Cl. G01N 25/20; G01N 1/44; G01N 33/00; G01N 35/00

(52) U.S. Cl. CPC G01N 25/20; G01N 1/44; G01N 33/0098; G01N 2035/00455

(57) ABSTRACT

A system and methods for non-destructive identification and separation of viability for gene bank conservation and/or varieties/variety groups of plant genetic resources, the system including at least: a space enclosed by walls; the temperature and humidity of which space can be controlled and which space contains one or more places suitable for measuring the plant genetic resources, suitable for placing trays for supporting the plant genetic resources; a thermal camera, which is positioned such that it faces the place suitable for placing one or more trays suitable for storing the plant genetic resources to be measured; a computer suitable for controlling the thermal camera and for storing and processing the recordings made by the camera; a unit for drying the plant genetic resources; a device for cooling and storing the plant genetic resources and one or more trays suitable for measuring the plant genetic resources.

### CROSS REFERENCE TO RELATED APPLICATION(S)

This application is a 35 U.S.C. § 371 National Stage of International Patent Application No. PCT/HU2024/050005, filed Jan. 29, 2024, claiming benefit from Hungarian Patent Application No. P 2300042, filed Jan. 30, 2023, the disclosures of which are incorporated herein in their entirety by reference, and priority is claimed to each of the foregoing.

### THE FIELD OF THE INVENTION

The present invention relates to a system for the identification and separation of the viability for gene bank conservation of plant genetic resources and/or their varieties/variety groups within the field of agriculture, which is suitable for a non-destructive (non-invasive) testing of plant genetic resources. The present invention further relates to a method for the identification and separation of varieties/varieties of plant genetic resources and to the use of the above-mentioned system and method irrespective of the method by which the variety is produced (by traditional breeding method or biotechnology or any other method).

### BACKGROUND OF THE INVENTION

In order for our Earth to function and humanity's access to food, it is essential to preserve all elements of the flora, including wild plant species and cultivated plants. In addition to preservation, sustainable use is key, as provided for in the convention adopted by the United Nations in June, 1992 (Biological Diversity Convention). Sustainable use, according to the aforementioned, is defined as “the use of components of biological diversity in a way and at a rate that does not lead to a long-term decline of biological diversity, thereby maintaining its potential to meet the needs and aspirations of present and future generations”. The Food and Agriculture Organization of the United Nations (FAO) has drawn attention on several occasions to the fact that there is a decline in the diversity of plant genetic resources for food and agricultural purposes, according to the data, more than three quarters of the varieties used in agriculture have gone out of cultivation (FAO SoWPGR-2, 2010, The second report on the state of the world's plant genetic resources for food and agriculture, <https://www.fao.org/3/i1500e/i1500e00.pdf>). In other words, the genetic preservation of wild and cultivated plant species in genebanks plays a significant role in ensuring long-term food security worldwide. The conservation of field crop seeds in refrigerated storage began worldwide in the 1970s. The methods and conditions of genebank conservation are laid down in detail in Hungarian (Genebank Standards 1994) and international (FAO 2013) genebank standards. At present, seedbank storage temperatures are generally −18° C.±3° C., at 4-6% humidity, using moisture-proof packaging material, so that the life expectancy is over 100 years (for cereals and legumes) (FAO; Genebank Standards. UN Food and Agriculture Organization, Commission on Genetic Resources for Food and Agriculture, 14th Regular Session, Rome, 2013). Several methods are available to conserve plant genetic resources. For arable crops and vegetable crops, the most common method is seedbank conservation. In addition, for certain groups of crops (fruit, vines, certain other perennial crops), in-stock genebank conservation is the primary method (genebank plantations). In addition, a number of novel methods are available for the conservation of plant genetic resources (in vitro meristem culture, cryopreservation, etc.) .

The key in seedbank conservation is to store enough amount of seeds. Indeed, seedbank conservation consists of several tasks to be performed. In addition to conservation, the basic tasks of all genebanks include documentation, multiplication/replication sowings, propagation and viability testing using standardised methods. The viability test most commonly used by genebanks is based on the germination of seeds, after which the germinated seed can no longer be stored as a genetic resource, i.e. the amount of stored seeds is reduced during these tests. A sufficient amount of seeds for genebank conservation is usually 1500-2000 seeds, but genebanking practice may differ in both positive and negative ways. Viability testing is necessary in genebank conservation because non-viable seeds or those that germinate poorly are not suitable for the conservation of genetic diversity and would lead to the deterioration of the genetic stock of the variety (WALSH, D. G. F, WALDREN, S., MARTIN, J. ; Monitoring seed viability of fifteen species after storage in the Irish Threatened Plant Genebank. Biology and Environment-proceedings of The Royal Irish Academy; 2003, 103 (2), 59-67.) We note that germination results alone do not always give a direct indication of viability. At the same time, in general, germination is still considered the most important viability test method according to genebank protocols, even though we know that viability does not directly imply germination ability (TIHANYI Z., TOMPA K. ; Forest breeding and cultivation of propagating material: practices. University of Forestry and Wood Industry, Sopron, 1985, 209.). In addition to germination, there are a number of viability testing methods available and their number is increasing year by year as science and technology progress.

According to the state of the art, several devices and methods for viability testing are known for testing plant genetic resources, which essentially means genebank testing of seed lots.

The apparent viability test is associated with Zelenchuk's name, during which seeds that appear intact are subjected to moderate pressure with a finger, needle or other instrument, and if the seeds resist, they are considered viable. However, the use of this method is based on rather subjective grounds (ZELENCHUK, T. K. ; The content of viable seeds in meadow soils of the L'vov Region. Byull. Mosk. Ova Ispyt. Prir., Otd. Biol., 1961, 66 (3), 77-92.).

Pruning is used for large-seeded species of forestry importance, mainly to identify seed health. This method can be used to separate weak, abnormally developing, infected seeds (SUSZKA, B., MULLER, C., BONNET-MASIMBERT, M., The seeds of deciduous forest trees from collection to sowing. Mezőgazda Kiadó, Budapest, 2008, 291).

The ISTA Tree Seed Committee [ISTA (International Seed Testing Association), 2008, International rules for seed testing. Bassersdorf, Switzerland (“unofficial translation”: International Seed Testing Association, 2008, International Rules for Seed Testing, Bassersdorf, Switzerland) ] has developed a method primarily for identifying the viability of tree seeds. The method consists of removing the embryos from the fruit or seed coat after soaking and keeping them in conditions that are suitable for germination and long enough for signs of non-viability to show (incubation). A viable embryo is one that is growing or remains fresh and intact until the end of incubation.

The measurement of glutamic acid decarboxylase activity (GADA test) for identifying viability was developed by Ellis et al. The essence of their method is that under the influence of the enzyme glutamic acid decarboxylase, the healthy seed produces carbon dioxide, the amount of which can be measured. The seed sample with the higher amount of carbon dioxide produced has the higher viability (ELLIS, R.H., HONG, T.D., ROBERTS, E.H. ; Handbook of seed technology for genebanks. Handbooks for Genebanks n. 2, International Plant Genetic Resources Institute, 1985a, 210; ISBN: 978-92-9043-118-3, ISBN: 92-9043-118-0); ELLIS, R.H., HONG, T.D., ROBERTS, E. H. ; Handbook of seed technology for genebanks. International Board for Plant Genetic Resources. 1985b, 456. ; ISBN: 978-92-9043-119-0, ISBN: 92-9043-119-9).

In the case of vital staining, the detection of viability is based on the fact that large molecular dyes (such as indigo carmine, fuchsine, Evan blue) cannot penetrate living cells at all due to the impermeability of the boundary membranes. Dead cells with damaged cell walls and cell membranes, on the other hand, are stained, as the dye molecules easily penetrate them. Nyebuljov (1925) was the first to use indigo carmine to demonstrate viability and to infer viability on this basis (NYEBULJOV D.N. ; On the methods of determining germination capacity without germination testing. Jard. Botan. Inst. Essias Semences Leningrad, Ann. Essais Semences 1925, 7, 31-35.).

Lakon (1942) built on Nebuljov's aforementioned method who applied it in the tetrazolium method (LAKON, G. ;

Topographic proof of the germination capacity of cereal fruits by tetrazolium salts; Ber. Deut. Bot. Ges. 1942, 60. 299-305). The tetrazolium salts are used for the viability test, the method gives information about the state of respiration. In the presence of oxygen, living cells reduce various solutions, so that tetrazolium salts can be regarded as redox indicators. The disadvantage of the tetrazolium method is that it is very difficult or impossible to perform on small seeds, does not detect dormancy and leads to the complete destruction of the tested plant unit.

Ivanov (1950) used a 0.1% acid fuchsin solution to detect viability [IVANOV, V. J., (1950)—Identification of the germination capacity of seeds by fuchsin. Selek. and Semen. 17. 60-61.].

Ivanov's aforementioned method was further developed by Effmann (1966) and Effmann and Specht (1967) by increasing the concentration of the fuchsin solution to 0.5%, thus reducing the identification time. Further time and labor savings have been achieved by batch sampling known as “sequence analysis” [EFFMANN, H., Application of the acid fuchsin method to the identification of the germination capacity of grasses, Thaer-Arch. 1966, 10., 205-212. ; EFFMANN, H., SPECHT, G., The family Poaceae (grasses) seed viability identification by acid fuchsin method, sequence analysis, Proc. Int. Seed Test. Ass., 1967, 32., 27-47.].

Gaff and Okong'o-Ogala (1971) used solutions of different concentrations of Evan's blue to identify viability (D.F., OKONGO-OGALA, O. ; The use of non permeating pigments for testing the survival of cells; Journal of Experimental Botany; 1971, 22, 756-758).

X-ray radiography is recommended for the detection of viability and insect damage of woody plants (e.g. Carpinus betulus, Fraxinus spp.) that germinate with difficulty or for a long time (SZABÓ LÁSZLÓ GY. (ed.) ; Fundamentals of Seed Biology. Akadémiai Kiadó, Budapest, 1980, 391. ) The method is rapid and does not damage the seeds. The test is performed by placing the seeds on a calibrated plate over a film. The irradiation lasts for a short time, usually a few seconds, the duration being determined by the wood species, the thickness of the seed and the sensitivity of the film. A test using a contrast medium is also common. The embryonic tissues of dead seeds absorb contrast agents that are impermeable to X-rays. Living cells are not penetrated by contrast agent (SMITH, R. D., DICKIE, J. B., LININGTON, S. H., PRITCHARD, H. W., PROBERT, R. J., EDS. ; Seed conservation: turning science into practice; Chapter 24. Kew, UK, Royal Botanic Gardens, 2003; SUSZKA, B., MULLER, C., BONNET-MASIMBERT, M., Seeds of deciduous forest trees from collection to sowing. Mezőgazda Kiadó, Budapest, 2008, 291. ).

Patent application No. WO08150798A1 describes an automated, contamination-free procedure for sampling and testing seeds and the related system. The system consists of a seed loading station, an imaging station to collect image data about the seed, and a seed orientation station to independently position and hold each seed in the desired orientation. The method according to WO08150798A1 cannot be considered non-destructive, since a tissue sample is taken from each individual seed.

A sampling procedure for seed tissue is described in patent application No. EP3097398A1, which is suitable for genotyping. According to the description, despite the tissue samples taken from the seeds, the germination capacity of the seeds can be preserved with the method. Tissue samples are taken by manual or semi-automatic perforation of the tested seeds.

An automated system for taking tissue samples from seeds is described in patent application No. WO18236874A1. The tissue sampling of seeds is performed in such a way that the seeds retain their germination viability, however, the testing method is not considered non-destructive precisely because of the tissue sampling. According to the description of patent application No. WO18236874A1, genotyping is the method of identifying which genetic variants are present in the tested seed. No mention is made of a thermal camera procedure to distinguish varieties from each other, the test according to WO18236874A1 is clearly based on the examination of the DNA of the seeds.

The viability of dormant seeds is examined using infrared (IR) contrast imaging in patent application No. US2020267890AA. The seeds are heated using spectral illumination, so that contrasting IR images are obtained, which makes the metabolism of the embryos of the seeds visible. From the presence of CO₂ emissions resulting from the metabolism of the plant seed, it can be deduced whether the seed is in an active, i.e. viable, state (the CO₂ band gives a characteristic sign on the IR spectrum).

Patent application No. WO18015495A1 deals with the prediction of the germinability of maize seeds by NMR. The steps of the method are as follows: a) measurement of NMR parameter(s) on maize seeds, b) prediction of germination profile based on NMR measurement, application of a mathematical model.

In patent application No. WO2020009978A1, it is mentioned that seed viability is traditionally carried out in a laboratory setting using human power. Patent application No. WO2020009978A1 describes an automated seed planting and evaluation system. The automated system allows errors due to human labour to be eliminated. According to the description, the seeds are planted in all cases, which means that the method cannot be called non-destructive.

Patent application No. IN202231013203 uses an artificial intelligence (AI)-based computing system to identify seed quality and germination patterns.

Seed imaging and related method are covered by patent application No. WO19178238A1. The imaging and analysis unit (designated as reference 14 in the description) collects image data of the seed under examination and identifies the characteristics of the seed (colour, size, shape, texture, internal composition, weight, volume, moisture content and chemical composition) using optimised image analysis algorithms. It is stated in the description that by combining two or more imaging modalities, a more accurate prediction of seed quality can be given. Seed viability is investigated in the context of seed surface contamination, diseases, composition in patent application No. WO19178238A1.

Flowchart 2B of patent application No. WO2019173606A1 shows a block diagram of a method for assessing seed viability. The method uses spectral images of seeds to infer which seeds are worth sowing. The description of patent application No. WO2019173606A1 is revealed that spectral imaging devices include multi- and hyperspectral imagers. The spectral images and the viability value of the seeds deemed viable are stored in a database, which could be used for spectral code search and/or artificial intelligence-based pattern recognition algorithm.

Patent application No. WO2016084452A1 describes a method for selecting seeds of coniferous plants. The steps of the method are as follows: a) irradiating the seeds with near infrared light, measuring the spectrum of near infrared light reflected from each seed, and obtaining reflectance data at each wavelength; b) obtaining reflectance data in the range 750 to 2200 nm at two specific wavelengths; c) calculating the ratio of the recoverable reflectance values for each seed; and d) comparing the ratio calculated in c) with the threshold value for the ratio of reflectance values at the two specific wavelengths to identify whether the seed has a germinating embryo, where i) the threshold value is used to test whether the seed has a germinating embryo. No reference is made in the description to the measurement parameters, nor is the sample preparation explained, and seeds of non-grain/agricultural crops are examined.

Tests related to seed viability are described in Example 1 of patent application No. WO09128998A1. The tests involve destruction, that is, the actual germination of the seeds also takes place.

The invention described in patent application No. WO2012048897A1 relates to a method for classifying seeds based on IR spectrum. In paragraph [0085] of patent application No. WO2012048897A1 it is described that in an embodiment of the invention IR spectra, in particular near IR spectra, are used to identify free spaces within seeds, but no mention is made in the description of segregation of varieties/variety groups and testing of seed viability.

A method and system for modelling seed structure is described in patent application No. WO13012860A1, which identifies the seed/seedling seed structure morphology by spectral analysis. In the tray shown in FIG. 1, the seeds to be tested are placed one by one in the sample containers. The tray is placed in the analyser by a robotic arm. The seed is incubated before measurement, one way of doing this is to soak it in water before measurement. Furthermore, according to the description in WO13012860A1, the main focus is on the classification of the seeds rather than on the non-destructive viability test.

A method and device for non-destructive testing of seeds is described in patent application No. US2015135585AA. Patent application No. US2015135585AA lists the following in relation to non-destructive measurements: ‘not exclusively, NIR, IR, NMR, X-ray, hyperspectral, UV and RGB imaging’. Sampling that does not affect viability is also necessary in this case, so the method cannot be considered 100% non-destructive.

The method set out in patent application No. WO10000266A1 is also used for the classification of seeds according to quality. The grading procedure comprises the following steps: a) obtaining the optical spectrum of the seed in one or more spectral ranges, and b) identifying the seed quality category by comparing the data of step a) with the reference spectra. The method is particularly suitable for the classification of seeds of pines of the genus Abies.

A method for identifying the maturity and quality of seeds by electromagnetic radiation is covered by patent application No. WO199742489A1. The irradiation (the source can be LED or laser) causes the chlorophyll in the seed to produce a detectable fluorescent signal. The invention also relates to a device for sorting seeds.

Patent application No. WO2001089288A1 relates to the classification of seeds. The classification is based on spectral data recorded from the seed. The spectral data obtained are compared with the reference spectral data and used to infer viability. Prior to measurement, the seeds are pre-treated with water and partially dried. The seeds are irradiated during measurement in the wavelength range 180-2500 nm, i.e. the irradiation method is used here to classify the seeds.

Patent No. EP1188041B1 relates to a method for identifying the germination capacity of seed grains, in particular cereals. In step 1, the seed grains are irradiated with light and at the end of the irradiation method at least one characteristic value of the light emitted by the seed grains is measured; in step 2, at least one characteristic value of the light emitted by the seed grains without external light stimulation is measured; from this characteristic value(s) the germination capacity is inferred. Although this prior art document examines viability, it does so under the influence of external stimulation (irradiation).

A physical measurement methodology for the differentiation of genetically modified maize from non-modified maize was presented at the IV Conference on Ecotoxicology in 2014 (József Berke, Hajnalka Bánáti, Borbála Baktay, Ottó Szalkovszki, Rita Szabó, Eszter Takács, Béla Darvas, Ferenc Gyulai; Possibilities of separation by thermal imaging on progeny seeds of maize variety MON 810 BT; IV. Ecotoxicology Conference; November 2014). During the presentation, it was concluded that with the further development of the method, in the case of maize, the separation of groups within the main classes is expected.

Kranner et al. examined seeds with a thermal camera and deduced viability. They found that infrared thermography can detect the biophysical and biochemical changes associated with moisture uptake and germination. The cooling phenomenon observed in the initial phase of the thermal profile is explained as a consequence of the dissolution of low molecular weight carbohydrates. According to Kranner et al., the kinetics of the production of such “cooling” components varies over time as a function of seed viability (KRANNER, I., KASTBERGER, G., HARTBAUER, M., PRITCHARD, H. ; Noninvasive diagnosis of seed viability using infrared thermography; Proceedings of the National Academy of Sciences of the United States of America; 2010, 107. 3912 7.

10.1073/pnas.0914197107.).

In connection with the announcement it is important to note that the “cooling effect” observed during the evaluation of the measurement results was, in our opinion, caused by the precipitated vapour on the surface of the seeds, i.e. it is due to a measurement error, and therefore the measurement method cannot be considered reliable, as the measurements were not performed under constant environmental conditions.

Men et al. performed viability studies on peas using thermal imaging (MEN, S., YAN, L., LIU, J., QIAN, H., LUO, Q. ; A Classification Method for Seed Viability Assessment with Infrared Thermography; 2017, Apr Basel 12; 17(4), 845. doi: 10.3390/s17040845. PMID: 28417907 Free PMC article.). Seed samples were individually examined in thermal and visual ranges every 5^{th} minute at 24° C. during 5 days of germination. That is, in this case, germination was also performed and the measurement method was time consuming.

Fernandez et al. studied peas and lichens under controlled conditions (FERNÁNDEZ-MARÍN, B., BUCHNER, O., KASTBERGER, G., PIOMBINO, F., GARCÍA-PLAZAOLA, J. I., KRANNER, I. ; Non invasive diagnosis of viability in seeds and lichens by infrared thermography under controlled environmental conditions; Plant Methods; 2019 December 5;15:147.). The measurement was carried out in an incubator with adjustable humidity and gas composition (relevant for lichens). In the thermal range, the “thermal fingerprints” of pea seeds were observed at relative humidity levels of 30 and 60% for 96 hours during water soaking. The seeds were then germinated out of the measuring chamber while being placed on wet filter paper under the IR camera. The infrared images were recorded at a rate of 1 frame per minute for 4 days for seeds and 1 frame per second for 170 minutes for lichens. Humidity was monitored continuously throughout the measurement. However, their measurements were taken over a long period of time, recording virtually the germination (water uptake) process for 96 hours in the case of seeds. Thus, on the one hand, the seeds tested can no longer be used for further analysis, and on the other hand, these seeds are considered lost from a genebank point of view.

ElMasry et al. provide an overview of research on the use of thermal imaging systems to seeds, including estimation of seed viability, detection of fungal and insect damage, investigation of seed damage and contamination, and seed varietal identification and classification. The article emphasises that measurements using thermal imaging systems are non-contact and therefore do not damage the seed being tested, unlike laboratory destructive tests, which are also more time-consuming and labour-intensive. The authors of the article investigated the biochemical/biophysical characteristics of pea and lettuce seeds, and were able to identify the difference between healthy and aged seeds from the water uptake of the seeds and the rate of degradation of their reserve nutrients. Healthy and aged seeds were tested during 24, 48, 72, 96, 120, 144 and 168 hours. The methods mentioned in this publication are only tangentially described, so the full, specific method of measurement is not included (ELMASRY, G., ELGAMAL, R., MANDOUR, N., GOU, P., AL-REJAIE, S., BELIN, E., ROUSSEAU, D. ; Emerging thermal imaging techniques for seed quality evaluation: principles and applications; Food Res Int. 2020 May; 131, 109025. doi: 10.1016/j. foodres. 2020. 109025. Epub 2020 January 22. PMID: 32247450 Review.).

In their article, Liu et al. state that seed viability testing is greatly facilitated by the method of non-invasive, high-resolution infrared thermography (LIU, L., WANG, Z., LI, J., ZHANG, X., WANG, R. ; A Non-Invasive Analysis of Seed Vigor by Infrared Thermography. Plants (Basel) ; 2020 June 19; 9(6 ):768. doi: 10.3390/plants9060768. PMID: 32575514 Free PMC article). Siberian elm seeds were collected from 30-year-old trees, dried at room temperature for 4 days, and then stored in nylon bags at −20° C. until the test. The following species were also examined: Chinese prickly pear, white acacia, soybean, rice, maize, tomato, the seeds of which were stored at 4° C. for approx. for 1 year. Siberian elm was tested after 0, 24, 48, 72, 96 and 120 hours. The seeds were artificially aged for the experiment and then germinated, during which thermal camera images were taken, so the method cannot be called non-destructive.

Thakur et al. investigated the “infrared thermal fingerprints” of onion seeds (THAKUR, M., SHARMA, P., ANAND, A., PANDITA, VK., BHATIA, A., PUSHKAR, S. ; Raffinose and Hexose Sugar Content During Germination Are Related to Infrared Thermal Fingerprints of Primed Onion (Allium cepa L.) Seeds; Front Plant Sci. 2020 October 6; 11:579037.). The measurements were carried out for 8, 12, 16, 20, 24, 28, 32, 36 and 52 hours while maintaining almost constant environmental conditions, which makes the method very expensive.

According to the state of the art, there is no known system for identifying the viability of seeds and the corresponding method, which could provide more accurate information about the viability of the seeds than the methods according to the state of the art, without damaging or stimulating them externally, which could reduce the amount of seeds used for this purpose during genebank studies, and is also suitable for quality assurance tests of commercially available seeds. Furthermore, there is no known system and method suitable for testing seeds, which, in addition to testing viability for gene bank conservation, is also suitable for identifying and separating varieties/groups of varieties, also non-destructively.

Due to the limited amount of seeds stored in genebanks, it is necessary to use non-invasive, non-destructive viability tests, since the examined seeds can be stored in the genebank even after such tests, and the tests can be repeated with the same seeds. Therefore, procedures using physical effects (incision, pressing, etc.) and chemical substances (paints) or radiation should be avoided, since after the application of such methods, the tested seed is no longer suitable for genebank storage. Non-invasive and non-destructive tests that require longer time should also be avoided, as such tests should not be carried out at the genebank storage temperature (typically −20° C.) but at temperatures higher than that, however, irreversible life processes start in the examined seed over a longer period of time, which it does not allow the continuation of genebank conservation after such an examination, the re-placement of the seeds after drying into refrigerated seed storages. In addition to viability tests, a key issue in genebank practice is also the identification and separation of varieties and groups of varieties, for which the state of the art only provides solutions involving destruction and morphological separation methods in the open field.

The aim of the present invention is therefore to provide a system for identifying the viability for gene bank conservation of seeds and a related method, which is free from the above problems of the state-of-the-art solutions and which is also suitable for identifying and separating varieties and groups of varieties. Our goal was to develop a new method and a system for implementing it, with the help of which we can get a more accurate picture of the viability of seeds stored in the genebank and other seeds than is currently available in the state of the art, would not damage the seed under examination and, once implemented, would remain suitable for further genebank conservation and further testing.

We have recognized that if seeds stored in a genebank (e.g. at −20° C.) are measured using a thermal camera testing system, we can identify the viability of the seeds with high accuracy and without destroying the seeds, so that the tested seeds remain suitable for further genebank conservation. It is also recognized that this system and this method is suitable for identifying the viability of plant genetic resources in addition to identifying and separating varieties/groups of varieties, irrespective of the method by which the variety was developed (traditional breeding methods or novel biotechnological methods). It is further recognized that the method and system according to the present invention is suitable for testing not only seeds but also other plant genetic resources.

The present invention is therefore essentially based on the unexpected realization that the system according to the invention for testing the viability for gene bank conservation of plant genetic resources, preferably seeds and/or for identifying and separating varieties/groups of varieties, and the associated method can non-destructively identify the viability for gene bank conservation of plant genetic resources, preferably seeds and/or varieties/groups of varieties to be identified and separated and to provide reliable data about them, furthermore the tested plant genetic resource, preferably seed, remains suitable for further genebank conservation, for other tests including further viability tests. The basis of the system for testing the viability of plant genetic resources, preferably seeds, and/or for the identification and separation of varieties/groups of varieties, and the related method according to the invention is that thermal camera images are taken of the plant genetic resources to be tested, preferably seeds, during their short-term heating and the measurement parameters are chosen in such a way that the performed measurements are reliable.

In other words, the essential advantage of the solution according to the present invention compared to the state-of-the-art systems for the viability of seeds and/or the separation of varieties/groups of varieties and their associated methods is that the system and method according to the invention for identifying and separating the viability of plant genetic resources, preferably seeds and/or its varieties/groups of varieties, provides reliable results for plant genetic resources, preferably seeds, both in terms of viability and the identification and separation of varieties/variety groups, all of this during a short measurement time, regardless of the plant species and the morphology of the seed, without lengthy sample preparation and germination, making it cost-effective, furthermore, the examined plant genetic resource, preferably seed, remains suitable for further conservation in the genebank.

### BRIEF DESCRIPTION OF THE FIGURES

FIG. 1 shows the average intensity-time graph recorded during the thermal camera examination of the sunflower achenes (FIR stands for Far Infrared, i.e. in the far infrared range), which clearly shows the three distinct warming stages (transient; steady; saturation stage).

FIG. 2 shows the average intensity curves of the thermal camera measurements of full and empty sunflower achenes as a function of time, the curves of the two types of achenes clearly separate from each other, thus the viability of a sunflower achenes of unknown viability can be easily identified.

FIG. 3 shows the average intensity-time graph taken based on thermal camera measurements of the seeds of the common bean.

FIG. 4 shows the average intensity-time graph taken based on thermal camera measurements of maize seeds.

FIG. 5 shows the average intensity-time graph recorded based on thermal camera measurements of the seeds of the einkorn.

FIG. 6 shows the average intensity-time graph of the non-germinated “discarded” and germinated seeds of common beans that were considered “discards”, recorded from thermal camera measurements.

FIG. 7 shows the average intensity-time graph of hybrid and genebank maize seeds measured with a thermal camera for the purpose of separating the variety groups.

### BRIEF DESCRIPTION OF THE INVENTION

The present invention relates to a system for the non-destructive identification and separation of the viability for gene bank conservation and/or varieties/variety groups of plant genetic resources, which system comprises at least:
a space enclosed by walls,
i) the temperature and humidity of which space can be controlled;
ii) which space contains one or more places suitable for measuring the plant genetic resources to be tested, suitable for placing trays for supporting the plant genetic resources;
thermal camera, which is positioned in such a way that it faces the place suitable for placing one or more trays suitable for storing the plant genetic resources to be measured;
a computer suitable for controlling the thermal camera and for storing and, where appropriate, processing the recordings made by the thermal camera;
a unit for drying the plant genetic resources to be tested;
a device for cooling and storing the plant genetic resources to be tested;
one or more trays suitable for measuring the plant genetic resources to be tested.

The present invention relates to the above mentioned system for the non-destructive identification and separation of the viability for gene bank conservation and/or varieties/variety groups of plant genetic resources, where the plant genetic resources are selected from the group comprising: seeds, other plant parts; the plant genetic resource is most preferably a seed.

According to a preferred embodiment, the drying unit of the system for the non-destructive identification and separation of the viability for gene bank conservation and/or varieties/variety groups of plant genetic resources is a drying chamber or other unit suitable for drying containing silica gel.

According to a further preferred embodiment, the thermal camera of the system for the non-destructive identification and separation of the viability for gene bank conservation and/or varieties/variety groups of plant genetic resources is arranged in an enclosed measuring space together with a location for storing one or more trays for the plant genetic resources to be measured, and/or the thermal camera is mounted on a support stand.

According to another preferred embodiment, the thermal camera of the system for the non-destructive identification and separation of the viability for gene bank conservation and/or varieties/variety groups of plant genetic resources according to the present invention has a spectral sensitivity of between 7 and 14 μm.

According to a preferred embodiment of the system for the non-destructive identification and separation of the viability and/or varieties/variety groups of plant genetic resources according to the present invention, the one or more trays suitable for measuring plant genetic resources have a split design.

According to a preferred embodiment of the system for the non-destructive identification and separation of the viability for gene bank conservation and/or varieties/groups of plant genetic resources according to the present invention, the material of the one or more trays suitable for measuring plant genetic resources is paper, suitable plastic, or any natural or artificial insulating material; the material of the one or more trays suitable for measuring plant genetic resources is preferably cardboard.

The present invention also relates to a method for the non-destructive identification and separation of the viability for gene bank conservation and/or varieties/variety groups of plant genetic resources, which method includes the following steps:
a) drying of the plant genetic resources to be tested;
b) cooling of said plant genetic resources after the drying according to step a) to a temperature of 0° C. or below;
c) setting of the temperature and humidity in the space for measurement, separated by walls;
d) placing of the plant genetic resources to be measured, cooled according to step b), under a thermal camera and measuring them with a thermal camera, where the measurement lasts no more than 2 hours;
e) software processing of the thermal camera recordings of the named plant genetic resources;
f) drawing a conclusion based on the result of the software processing obtained in step e) regarding the viability and/or variety/group of varieties of the measured plant genetic resources;
where named plant genetic resources are placed on one or more trays suitable for storing the plant genetic resources to be measured before step a), b) or c).

The present invention also relates to the above mentioned method for the non-destructive identification and separation of the viability for gene bank conservation and/or varieties/variety groups of plant genetic resources where the plant genetic resource is most preferably a seed.

According to a preferred embodiment of the invention, in step a) the temperature used for drying the plant genetic resources is between 10-25° C.

According to another preferred embodiment of the invention, in step a), the humidity is reduced during the drying of the plant genetic resources so that the relative humidity finally set between 10 and 15%.

According to a further preferred embodiment of the invention, in step b), the plant genetic resources are cooled for at least 24 hours.

In an even more preferred embodiment of the invention, the plant genetic resources to be measured are cooled to a temperature between 0° C. and −18° C.±3° C. in step b).

According to a further preferred embodiment of the invention, the plant genetic resources are cooled in a sealed vessel and/or on a tray suitable for measurement for the cooling according to step b).

According to an even more preferred embodiment of the invention, the measurement environment according to step c) has a temperature of 16° C.±4° C. and a relative humidity of 70-90%.

According to a preferred embodiment of the invention, in step d) the measurement time of the cooled plant genetic resources is up to 1 hour, preferably 30 minutes, most preferably 15 minutes.

According to a further preferred embodiment of the invention, the method is carried out with the system as described above.

The present invention also relates to the use of the above system for the non-destructive identification and separation of the viability for gene bank conservation and/or varieties/variety groups of plant genetic resources for the non-destructive identification and separation of the viability for gene bank conservation and/or varieties/variety groups of plant genetic resources.

The present invention also relates to the use of the above system for the non-destructive identification and separation of the viability for gene bank conservation and/or varieties/variety groups of plant genetic resources for the non-destructive identification and separation of viability for gene bank conservation and/or varieties/variety groups of plant genetic resources where the plant genetic resource is most preferably a seed.

### DETAILED DESCRIPTION OF THE INVENTION

The essence of the present invention is that with the system for identifying and separating the viability for gene bank conservation and/or varieties/variety groups of plant genetic resources non-destructively, and the associated method including a thermal camera described in detail below, the viability and/variety group/variety of the cooled plant genetic resources is identified and separated non-destructively in such a way that the tested plant genetic resource remains suitable for further genebank conservation.

Within the scope of the present specification if a numerical value is given, it is understood that the last digit of the given number shows the precision the given value in accordance with the rules of rounding. So, for example, 3.0 is understood to mean the range of 2.95-3.05.

For the purposes of this description, plant genetic resources are defined as all genetic material of plant origin, including reproductive and vegetative propagating material, which contains functional units of heredity, irrespective of the source of the plant genetic resource (traditional breeding techniques or novel biotechnological methods). Heritable material is defined as the whole genome of a plant, present in the plant in the form of DNA. For the purpose of this description, plant genetic resources are selected from the list of seeds, other parts of plants, preferably seeds. The seed is part of the flowering plant fruit that develops from the fertilized seedling, the organ that protects and nourishes the germ. Its components are usually the seed coat or fruit wall, the nutritive tissue (which may contain starch, oil or may be absent) and the germ.

Viability is generally understood as the property that a plant organism can withstand unfavorable conditions without significant damage. By examining the viability of plant genetic resources, we mean whether the examined plant genetic resource is viable, i.e. whether it is able to make its life processes active.

For example, there are countless viability testing methods for seeds. The most common method is germination. With this test, non-viable or poorly germinating seeds, which are not suitable for conserving genetic diversity, can be filtered out. At the same time, as we detailed in the description of the state of the art, we cannot always directly infer viability from the germination results alone due to seed dormancy and other factors. According to the genebank protocol, germination can be considered the most widespread viability test method, even though we know that viability cannot be clearly equated with germination capacity.

The prerequisites for germination of plant genetic resources are right temperature, moisture, light and presence of oxygen. If any of these conditions are not properly met, germination will not take place or will not take place properly. In addition, of course, many other factors influence the induction and progress of germination, but the four components listed above are the most decisive.

The first prerequisite for the initiation of germination is the amount of water required (SZABÓ LÁSZLÓ GY. (ed.) ; Fundamentals of Seed Biology; 1980, Akadémiai Kiadó, Budapest, 391. ). However, is difficult to achieve the optimum water supply of the germination medium. The ISTA (International Seed Testing Association) and AOSA (Association of Official Seed Analysts) standards provide extensive guidance on germination methods.

The identification and separation of the variety group or variety means the organization of individuals of plant genetic resources belonging to the same taxonomic category into one group and their separation from each other. The unit of cultivated plants is the variety (cultivar=cv.) . By variety, we mean a cultivated individual or group of plants belonging to the same species, from the point of view of the grower (farmer, gardener, forester) that share one or more essential characteristics, but differ from other species in at least one characteristic (Gyulai 1999). So far, the separation of cultivar groups and cultivars is possible only morphologically, with international descriptive tests (this requires the entire growing season and the entire plant) and/or DNA testing. Both morphological examination and DNA examination involve the loss of plant genetic resource.

The system according to the present invention and the related method are non-destructive, because not only is no tissue sample taken from the plant genetic resources, but also no external stimulation affects the plant genetic resources during measurement. The tested plant genetic resources can be used for further measurement, and we do not lose them from a genebank point of view.

The system for identifying and separating the viability for gene bank conservation and/or varieties/groups of plant genetic resources according to the present invention includes at least the following: a space delimited by walls, the temperature and humidity of which can be controlled and the space suitable for measuring the plant genetic resources to be tested also contains one or more places suitable for placing trays for supporting the plant genetic resources; a thermal camera positioned to face the one or more trays suitable for storing the plant genetic resources to be measured; a computer suitable for controlling the thermal camera and for storing and, where appropriate, processing the recordings made by the thermal camera; the unit for drying the plant genetic resources to be tested, the device for cooling and storing the plant genetic resources to be tested, and one or more trays suitable for measuring the plant genetic resources to be tested.

Within the framework of the present invention, the space delimited by walls is understood to mean the measuring laboratory serving as the location of the measurement. Walls are needed so that the temperature and humidity do not change to an extent that affects the measurement due to the movement of the ambient air during the measurement. It is preferable if all sides of the space delimited by walls are bounded by walls with the necessary doors and windows. However, the system also falls within the scope of protection of the present invention, where not all sides of the space delimited by walls, but only some sides are formed by walls, which are suitable for eliminating unfavorable air movements.

The temperature and humidity of the space separated by the walls need to be controllable. For a well-executed measurement, it is necessary to set the temperature appropriately, so that it falls within a specific temperature range, which temperature range depends on the plant genetic resource to be tested (e.g. 16° C. for seeds with a diameter greater than 3 mm). In the case of the present invention, a cooling-heating device, such as an air conditioner suitable for cooling and heating, is used to adjust the temperature. It is preferable to carry out the measurement in a space (measuring laboratory) separated by well-insulated walls, so that it is easier to maintain the temperature required for the measurement. We note that the temperature required for measurement can be set not only in a space separated by artificially insulated walls, but also, for example, in caves and hollows found in nature.

In addition to the temperature of the space delimited by the walls, its humidity must also be controllable, this is what the humidifying device is for, which is meant to ensure constant high humidity in the space delimited by the walls. As mentioned above, due to the moisture that precipitates on the surface of the plant genetic resources, in order to avoid incorrect measurement results, it is also important to maintain a constant measurement environment, one of which is to maintain constant humidity. Note that we distinguish the low relative humidity (10-15%) applied during drying detailed later in this description from the high relative humidity (70-90%) used during the measurement. On the one hand, the humidity required for measurement can be achieved artificially, for example by using humidification equipment, or we have to look for natural conditions where this condition exists (e.g. moist air in caves, mine passages, etc.).

As it is obvious to a person skilled in the art, the controllability of both temperature and humidity includes the measurability of these parameters, since the control can only be carried out by knowing the measured values. Of course, it is possible that the temperature and/or the humidity are just right for a specific measurement and it is not necessary to modify them with cooling-heating equipment or humidification equipment.

Part of the system for identifying and separating the viability for gene bank conservation and/or varieties/groups of plant genetic resources is a place suitable for receiving one or more trays for supporting the plant genetic resources suitable for measuring the plant genetic resources to be tested. This place is created under the thermal camera field of view and one or more trays can be placed on it for measurement.

Part of the system for identifying and separating the viability for gene bank conservation and/or varieties/variety groups of plant genetic resources is a thermal camera specifically suitable for carrying out such measurements (see the specification of the preferred thermal camera in detail below). The thermal camera is positioned in the space delimited by the walls so that it faces the one or more trays suitable for storing the plant genetic resources to be measured. With the help of the thermal camera, the physiological processes of the plant genetic resources can be examined, from which we can infer their viability, and based on the thermal camera recordings, the varieties/variety groups can be identified and separated.

In the case of the present invention, the thermal camera measurements are controlled by a computer, which is suitable for storing and, where appropriate, processing the recordings (image and video files) made during the measurements. The digitized images of the thermal camera measurements are processed using a software (e.g. the IRPlayer software version 4.0 (Hexium Kft, Budapest, Hungary) is suitable for this), which software can handle the unique file format of the thermal camera. The modules of the software we use are the following: reading image data; merging of files; application of a point measurement function; data conversion and data saving. In addition, the Lumi program was created specifically for thermal camera measurements (e.g. Lumi IDSF version 5.42, SFD Informatika Kft., Keszthely, Hungary), which is used for batch measurement of the intensity of digital images, based on fixed and individually defined linear intensity. Furthermore, with the help of a computer, the data from the data collector suitable for measuring temperature and humidity are extracted using a software (ComSoft Basic software Testo SE & Co. KGaA, Lenzkirch, Germany).

The drying unit is part of the system for the identification and separation of the viability for gene bank conservation and/or varieties/variety groups of plant genetic resources. Drying of plant genetic resources prior to measurement is important for several reasons. Firstly, dry plant genetic resources are less likely to form condensation on the surface, which would falsify the measurement results. On the other hand, seeds with low moisture content can be stored cooled to −20° C., as in this case the freezing of the water in the seeds, and thus the increase in volume, does not damage the cells.

A device for cooling and storing plant genetic resources is part of the system according to the invention, which can be a freezer or cold storage. The function of the device for cooling and storing plant genetic resources is twofold, on the one hand, it is used to cool plant genetic resources after drying, and it is also used to store plant genetic resources in a genebank.

Part of the system for identifying the viability for gene bank conservation and/or variety groups of plant genetic resources is one or more trays suitable for measuring genetic resources, which are placed in the space demarcated by the walls under the thermal camera field of view in the designated place suitable for carrying out thermal camera measurements. This one or more trays serve as the place of the plant genetic resources to be measured during the measurement, i.e. we place the seeds to be measured on them during the measurement. Consequently, the tray, on which placed the plant genetic resource to be measured (e.g. seed) and the thermal camera are positioned relative to each other so that the thermal camera can take a picture of the seeds resting on one or more trays. Thus, the thermal camera is positioned to face one or more trays suitable for storing the plant genetic resources to be measured.

A system for non-destructive identification and separation of plant genetic resources for viability for gene bank conservation and/or varieties/variety groups is used to test seeds and other plant parts, preferably seeds. These are the types of plant genetic resources most commonly stored in genebanks, and therefore the most frequent need is for viability testing of these types. By other plant parts, we mean fruits, parts of fruits and other reproductive plant organs.

The drying unit of a system for the non-destructive identification and separation of the viability for gene bank conservation and/or varieties/variety groups of plant genetic resources may be a drying chamber or other drying unit containing silica gel. Drying with silica gel can be carried out gently, at low temperature and for extended period without irreversible changes in the plant genetic resource. Drying is necessary for the reason explained above. According to our experience, the plant genetic resources to be tested can be dried most effectively in a drying chamber or, less frequently, in a drying unit using a drying agent (e. g. silica gel).

The thermal camera of the system for the non-destructive identification and separation of the viability for gene bank conservation and/or varieties/variety groups of plant genetic resources according to the invention is placed in an enclosed measuring chamber together with a place for placing the one or more trays for storing the plant genetic resources to be measured and/or the thermal camera is mounted on a support stand.

The humidity and temperature of the enclosed measuring space is essentially the same as the humidity and temperature of the space enclosed by walls, which is continuously measured and recorded during measurements (e.g. a sutible device for this the Testo 174 H data logger, Testo SE & Co. KGaA, Lenzkirch, Germany).

It is also possible to imagine an embodiment where the thermal camera is placed outside the closed measuring area, in which case the material that does not affect IR signal transmission must be chosen when choosing the material of the measuring area. However, it is preferable that the thermal camera or at least the objective of the thermal camera be located in the closed measuring space. In practice, a closed measuring room means a Plexiglas house with an openable window, the thermal camera placed in a closed measuring room is protected from external influences (for example, air movement, flow, the movement of researchers, etc.), by which our goal is to minimize factors that interfere with the measurement. The thermal camera required for the measurement is also placed on a table that is leveled and vibration-damped, thereby eliminating effects that disturb the measurement.

More precise measurement settings are possible by placing the thermal camera on an adjustable stand. Since the different plant genetic resources vary in size over a wide range of dimensions (can be mm or cm of the order of magnitude), they can all be monitored by the thermal camera of the system of the invention, regardless of their size, and no other recording system is needed for the tests.

In practice, the thermal camera of the system according to the invention detects electromagnetic waves in the infrared range. The thermal imaging camera of the system according to the invention preferably has a spectral sensitivity (i.e. an operating range) of 7-14 μm, which is designed to have a measurement range of −30° C. to 1000° C., to operate over a wide range of humidity (10-95%) and to perform automatic measurement corrections (outdoor temperature, distance, relative humidity). Further parameters related to the thermal camera preferably include:
Sensor type: uncooled FPA microbolometer
Zoom: ×1, ×2 (with digital zoom processing software)
Pixel count: 640×480
Built-in imaging devices: thermal camera (16 bit/pixel)
Outputs: LAN, external display connection
NETD (300K, 50 Hz) 30 mK
Power supply: external adapter (230V AC, 50 Hz).

The thermal sensitivity of the thermal camera is min. 25 mK, which is considered to be twice as sensitive as commercially available camera systems. This allows the detection of small temperature differences, which is essential to visualise the different temperature structural elements within plant genetic resources (e.g. seeds) and rapid thermal fluctuations. A further advantage of the thermal camera is the ability to adjust the angle of view to the size of the objects. Furthermore, the remote sensing system allows us to collect information about the object of study, i.e. the plant genetic resources, without any physical damage to them. Thus, by using a thermal camera, we can replace the traditional viability or germination tests, after which the plant genetic resources can no longer be used for further testing. The physiological parameters of the plant genetic resources are essentially unchanged during the thermal imaging, the impact of the thermal measurements on the plant genetic resources is negligible, so that their germination capacity is preserved in the subsequent course of the test, if the plant genetic resource was germinable before the test.

Before the thermal camera measurements, we perform calibration in all cases and, if necessary, between the measurements. We distinguish between two types of calibration, on the one hand, the sensor of the thermal camera can be calibrated, and on the other hand, the measuring area itself can be calibrated. The thermal camera records one image every 1-1.3 seconds.

We place the plant genetic resources on the one or more trays suitable for measuring the plant genetic resources in such a way that they do not roll, slide, or touch each other, and that there is a distance between them necessary for the measurement. The applied trays therefore preferably have an uneven surface, e.g. they are wrinkled, pleated, especially preferably split. This design of the tray makes it possible to measure several plant genetic resources at the same time.

During the design of the one or more trays suitable for measuring plant genetic resources, we examined a number of raw materials. During the testing, we monitored the temperature changes of the plant genetic resources, as well as the influencing effect of the media holding the plant genetic resources on their temperature. In addition, a material was needed for the trays that is workable to some extent, so it is possible to create a tray from it that is practical and suitable for the placement of plant genetic resources. During the testing, we examined seven different types of seed holding media: clay; glazed clay pottery; glass; foamed PVC (Palfoam™) ; baking plasticine; wood and paper.

During the thermal camera testing of the media holding plant genetic resources, i.e. the trays, we used the measurement procedure used for plant genetic resources, so we monitored the heating of the cooled plant genetic resources. The length of the test measurements depended on the type of medium. In total, we measured the media containing the individual plant genetic resources and the plant genetic resources placed on them for 5-45 minutes. The images taken by the high-sensitivity and uniquely developed thermal camera were saved as files with idsf extension, and then converted to .tif format for easier visual presentation later. In the case of glass, foamed PVC, clay and glazed clay pottery, only the material of the tray was measured without plant genetic resources, already from their measurement it became clear that the materials listed above are too good heat conductors, i.e. they are not suitable for the raw material of the tray. In the case of baking plasticine, during the measurements we found that the plasticine has a high heat capacity, which significantly affects the temperature change (warming) of the plant genetic resources placed on it, and accordingly reduces the reliability of the results of the physiological characteristics derived from the change in the temperature of the plant genetic resources. In addition, empirical studies have shown that in the case of baking plasticine, the length of the measurement increases, which can be attributed to the heat capacity of the medium that holds the plant genetic resources. During the measurements of pine wood planks, we found that, like plastic, the heat capacity of wood is also significant, which also greatly influences the temperature change (warming) of the plant genetic resources placed on it and, accordingly, the reliability of the results of the physiological characteristics derived from the change in the temperature of the plant genetic resources. In the case of pine wood, we also observed that the length of the measurement time increases, which, like the previous ones, can be attributed to the heat capacity of the medium that holds plant genetic resources. During measurements on paper, we found that while the heat capacity was high in the case of plasticine and pine wood planks, the heat capacity was lower in the case of paper, all of which can only have a minor effect on the temperature change (warming) of the plant genetic resources placed on it, and accordingly the reliability of the results of physiological characteristics derived from changes in the temperature of plant genetic resources. This was especially true for the cardboard tray. Based on the empirical tests, the length of the measurement time was short, unlike that of baking plastic and wood, which proves the use of paper as a medium for storing plant genetic resources and its adequate heat capacity. The split design of the paper tray was done so that the individual plant genetic resources could be physically separated from each other, all of which was achieved with the help of a grid. Based on the thermal camera recordings taken during the measurement, we clearly saw that the temperature of the paper tray did not change significantly even after five minutes and maintained a temperature value close to that of the test table. During the test measurements, paper-based cardboard proved to be the best among the tested tray raw materials. It is obvious to a person skilled in the art that thermal camera measurements can be performed on a tray made of any suitable material, which behaves similarly to a paper tray during measurement, i.e. during the thermal camera measurement, they do not significantly affect the heating of the seeds and are suitable for supporting the seeds. These are, for example, trays made of plastic or any natural or artificial insulating material suitable for thermal camera measurements.

The system for identifying and separating the viability and/or varieties/variety groups of plant genetic resources can be mobilized, the system can be installed in any laboratory as described here, be it a mobile shipping container or the laboratory of an institute dealing with plant resources (e. g. genebank) or a converted vehicle.

The present invention also relates to a method for identifying and separating the viability for gene bank conservation and/or varieties/variety groups of plant genetic resources.

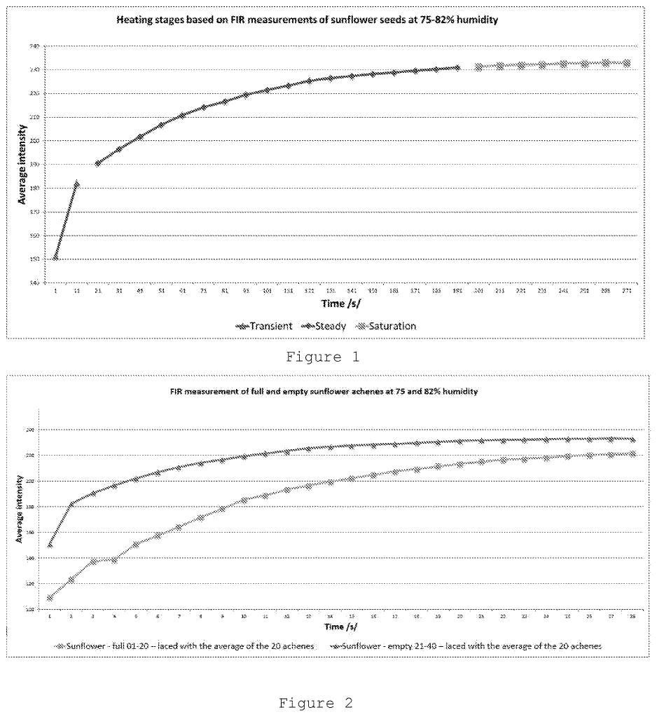

The method for identifying and separating the viability for gene bank conservation and/or varieties/variety groups of plant genetic resources includes the following steps:
a) drying of the plant genetic resources to be measured: during this step, the plant genetic resource from any species to be tested is dried at 10-25° C., depending on the variety, and the humidity is reduced to 10-15% relative humidity using a drying chamber or other suitable drying unit containing silica gel. We note that lower relative humidity from this damages the plant genetic resource. Plant genetic resources, depending on their type, have an average moisture content of 3-7% after drying.
b) After the drying according to step a), plant genetic resources are cooled to a temperature of 0° C. or below, the target temperature of the cooling depends on the plant genetic resource. The pre-dried plant genetic resources to be tested must be cooled in a cooling device for at least 24 hours before measurement. During the cooling, it is preferably cooled to −18° C.±3° C., the plant genetic resources required for the measurement are cooled in a closed container and/or on the one or more trays made of the paper or plastic used for the measurement, in a device for cooling, for example in a“no frost” freezer, that prevents the steam from condensing and freezing on the surface of the samples to be tested. The closed container can be hermetically sealed glass or a three-layer aluminum-plastic bag with a welded closure. During the measurement, special attention must be paid to the fact that dried plant genetic resources are often fragile and thus sensitive to mechanical influences, so they must always be handled with care, preferably avoiding shocks and bumps during their movement. The paper tray should only be touched on its edge when placing it in the freezer, thereby reducing the chance that the measurement results will be influenced by any external factors. We note that when placing the plant genetic resources on the tray, special care must be taken to ensure that the genetic resource to be measured and the tray are affected as little as possible by the heat impressions created by the touch of our hands/fingers.
c) Setting of the temperature and humidity to measure the viability of plant genetic resources and/or to identify and separate varieties/groups of varieties in the walled space. The measurement temperature is preferably between 16° C.±4° C., which is set using a cooling-heating device. A relative humidity between 70-90% is required to identify and separate the viability and/or variety/variety group, which values are continuously monitored with the appropriate measuring and data collection device.
d) Placing the batches of plant genetic resources to be cooled and measured according to step (b) under a thermal camera and measuring with a thermal camera, where the measurement lasts up to 2 hours: the prepared cooled plant genetic resources are carefully placed on the one or more trays made of paper or plastic with a dividing plate under the thermal camera, where the samples are measured in the visible and thermal range. The plant genetic resources are placed on the one or more trays made of divided paper so that they are spaced at an appropriate distance and do not interfere with each other during heating. Gloves should be worn when placing the tray under the thermal camera and throughout the measurement to prevent the warmth of the fingers from affecting the heating of the plant genetic resources. The plant genetic resources should not be touched before the measurement, if one of the samples to be measured has to be adjusted, this should only be done with a suitable instrument, e. g. tweezers. The duration of the measurement is variable, up to a maximum of 1 hour, preferably 30 minutes, most preferably 15 minutes. The reason for reducing the measurement time to 15 minutes is that the heating of the dried and cooled plant genetic resources is most intense in the first 5 minutes. It is in this 15-minute time interval that the plant genetic resources under measurement show the most significant and characteristic changes, after which their warming slows down. During the warming of plant genetic resources, we observed three distinct phases that are characteristic of all measured species: transient, steady, and saturation (see for example FIG. 1).

The transient phase is the initial phase of the heating of the cores, at which point the heating process of the cores begins. The section may have a different length for each measurement batch (note that here by measurement batches we mean plant genetic resources belonging to the same species/genebank batch), presumably due to transient processes (e.g. the speed of placing the tray, the initial temperature of the batches relative to each other) as well. The transient phase is on average between 4 and 10 seconds.

The length of the uniform warming phase is different for individual plant genetic resource species and presumably for cultivars. The length of the warm-up varies, on average it lasts 150-300 seconds.

In the saturation phase, the warming starts to be measured. The data of the line fitted to the saturation stage (slope, intercept, standard deviation) are typical for each plant genetic resource lot.

The most characteristic changes of the cooled plant genetic resources are shown when the temperature difference between the air and the plant genetic resource is the greatest, i.e. when the one or more trays are removed from the device for cooling, i.e. it is worth performing this operation as quickly as possible (we note that in the case of a closed measuring space the one or more trays can be inserted through the door formed on it). The determination of the 15-minute measurement time also played a role in ensuring that the measurement does not take too long in order to preserve the viability of the plant genetic resources, as well as that the plant genetic resources can still be stored after re-drying after the measurement, and that the measurement can be repeated. The international publications described in the state of the art section often write about measurements lasting longer than 24 hours, while the use of thermal camera technology is called a “non-invasive” method. However, in the case of such long measurements, the process of germination already begins (in many cases, therefore, the germination itself was monitored with a thermal camera), which means that after that, further examination of the same plant genetic resource and its use in the genebank is no longer possible, since it will become a seedling plant.
e) Software processing of the thermal camera images of the mentioned plant genetic resources: the Lumi IDSF 5.42 program is suitable for measuring the intensity of batch digital images based on a built-in and arbitrarily defined (so-called custom) function. In the Lumi IDSF 5.42 program, selection takes place on the entire surface of plant genetic resources, and the software measures the average intensity of the pixels belonging to the selected area. The intensity can be treated as the average of the emitted energy. This measured value is the digital value of the energy release per pixel. IRPlayer 4.0 is a software that manages the unique file format of the thermal camera, which also manages the image data built into the thermal camera during measurements, as well as image data created with external control software. It has five main modules: scan image data; merging of files; basic point measurement functions; choice of built-in palettes; data conversion, data backup.
f) drawing a conclusion based on the results of the software processing obtained in step e) regarding the viability and/or variety group of the measured plant genetic resources: During the evaluation of the measurement results, average intensity-time graphs are obtained, from which a conclusion can be drawn regarding the viability and/or varieties/variety group of the measured plant genetic resources;
   where named plant genetic resources are placed on the one or more trays suitable for storing the plant genetic resources to be measured before step a), b) or c).

In the case of a viability test for gene bank conservation, viable plant genetic resources were germinated to verify the thermal camera measurement results. Germination was necessary solely to verify the results of the thermal camera measurements, i.e. it is not part of the steps of the procedure. Our objective remains to identify the viability for gene bank conservation of plant genetic resources using a non-destructive method. In each case, the control germination of the plant genetic resources was carried out according to the standard applicable to the given species.

The following genetic resources can preferably be examined with the method for the non-destructive identification and separation of the viability for gene bank conservation and/or varieties/variety groups of plant genetic resources: seeds, other plant parts, where under other plant parts we understand fruits, fruit parts, other reproductive plant parts, etc. Most preferably, the plant genetic resource is a seed.

The method according to the present invention detailed above is preferably carried out with a system for the non-destructive identification and separation of the viability for gene bank conservation and/or varieties/variety groups of plant genetic resources.

The system for identifying and separating the viability for gene bank conservation and/or varieties/variety groups of the plant genetic resources according to the present invention is preferably used for the non-destructive identification and separation of the viability for gene bank conservation and/or varieties/variety groups of the plant genetic resources.

The present invention also applies to the non-destructive identification and separation of the viability for gene bank conservation and/or varieties/variety groups of plant genetic resources of the system for the non-destructive identification and separation of the viability for gene bank conservation and/or varieties/variety groups of plant genetic resources, where the plant genetic resources are preferably selected from the group comprising the following: seeds, other plant parts, where the latter is taken to mean fruits, fruit parts, other reproductive plant parts, etc. The plant genetic resource is most preferably a seed.

### Example 1: Separation of a Empty and Not-Empty Sunflower Achenes-Viability Test

a) Before measurement, sunflower achenes were dried in a drying chamber at 20° C. starting from a relative humidity of 20-22 % with a continuous decrease in relative humidity (reaching and maintaining a relative humidity between 10-15 %) to a seed moisture content of 3.9 %.
b) The sunflower achenes dried according to step a) were cooled for at least 24 hours at −18° C.±3° C. on the tray made of divided cardboard used for measurement (we placed 20 achenes per tray) in a “no frost” freezer.
c) The temperature of the walled space (measuring laboratory) was set to 16° C., the measured relative humidity was between 75-82.2%.
d) As soon as possible, the tray containing the sunflower achenes cooled according to step b) was removed from the freezer and placed under the thermal camera and the thermal camera measurement was started. The measurement time was 15 minutes.
e) We processed and evaluated the thermal camera images of the sunflower achenes using software.
f) Based on the results of the software processing, we drew a conclusion regarding the viability of the sunflower achenes.

In order to support the reliability of the procedure, we germinated the sunflower achenes.

The achene of a sunflower is “full” if there is a seed in it and “empty” if there are no seeds in the achene, i.e. the achene is empty. We measured the achene of 20-20 sunflowers in four series. We measured the heating of sunflower achenes for 15 minutes, but the first 300 seconds of the measurements were the most interesting, as this is where the achenes belonging to each group showed intense separation.

The far-infrared (hereinafter FIR) curves for full and empty sunflower achenes differ significantly on average and per seed (min. three-fold standard deviation). Empty sunflower achenes are in principle saturated with air, full achenes in principle contain seeds. During the measurements, the aim was to separate empty and full sunflower achenes based on thermal camera measurements. Afterwards, the seeds were germinated as a check. After the genebank propagation, the empty achenes were selected for measurement using a separating machine based on specific gravity (“winding”).

The slope of the initial transient stage is higher in the case of empty sunflower achenes, because the interior of the achene is filled with air, there are no seeds in it. The second, uniform heating stage is almost invisible for empty seeds, while it is clearly visible for full seeds. The third, saturation stage is steeper for full seeds (see FIG. 2). In FIG. 2, the curves showing the average intensity of full and empty seeds are clearly separated from each other depending on time. We note that in FIG. 2, for easier interpretation, the average intensities are plotted as a function of the first 28 seconds of the measurement. From these results, in the case of sunflower, it is possible to clearly infer the expected viability, since the seeds considered to be empty are usually not able to germinate. The unknown achene can be classified based on the processed data of the thermal camera measurement, the course of the curve belonging to the given sunflower achene and the corresponding values (average intensity) are unique, that is, it can be identify whether the measured values belong to the full or empty achene.

### Example 2: Thermal Camera Viability Test of Common Bean, Maize and Einkorn

a) Prior to measurement, the seeds/grains of common beans, maize and einkorn were dried in a drying chamber at 20° C. starting from a relative humidity of 20-22% with a continuous decrease in relative humidity (reaching and maintaining a relative humidity between 10-15%). Using this method, we achieved a seed moisture content of 6.9% in the case of the einkorn and 6.1% in the case of maize and common beans. We note that the seeds/grains of the common beans, maize and einkorn were selected for the test measurement in such a way that out of 100 seeds/grains per species, 50 seeds/grains were considered discards from the genebank point of view, and 50 seeds/grains were viable, which we were sure about based on previous control germination results.
b) The dried common beans, maize and einkorn seeds/grains according to step a) were stored for 24 hours at −18° C.±3° C. on the tray made of divided cardboard used for measurement (we placed 20 seeds/grains per tray, a total of 100 seeds/grains of each species we measured), cooled in a “no frost” freezer.
c) The temperature of the space (measuring laboratory) separated by walls was set to 14-15° C., the relative humidity to 78.6-88%, which values were continuously monitored during the measurement.
d) The trays containing the seeds/grains of common beans, maize and einkorn cooled according to step b) were taken out of the freezer and placed under the thermal camera as soon as possible and the thermal camera measurement was started. The measurement time for each measurement item (tray) was 15 minutes.
e) We processed the thermal camera images of the seeds/grains of the common beans, maize and einkorn with software.
f) Based on the results of the software processing, we drew conclusions regarding the viability of the above-mentioned plant genetic resources.

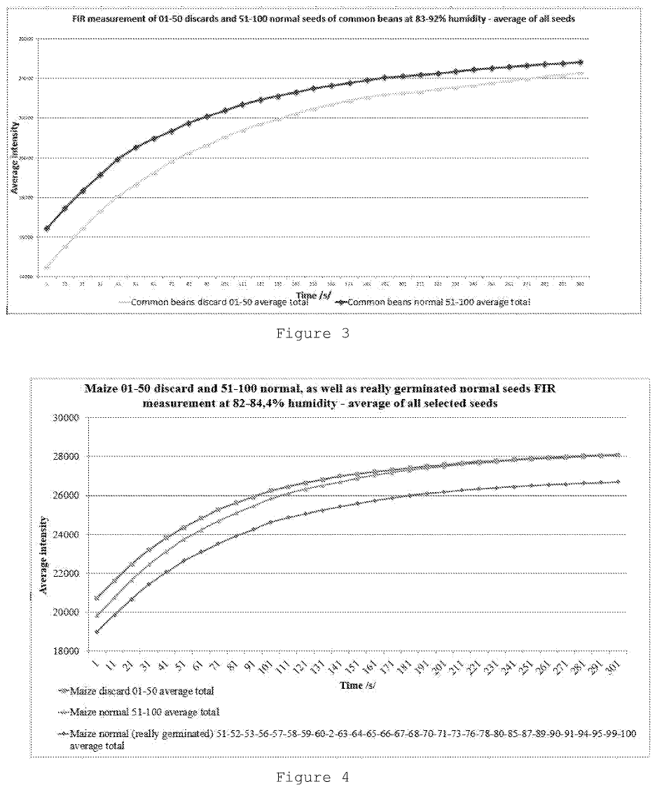

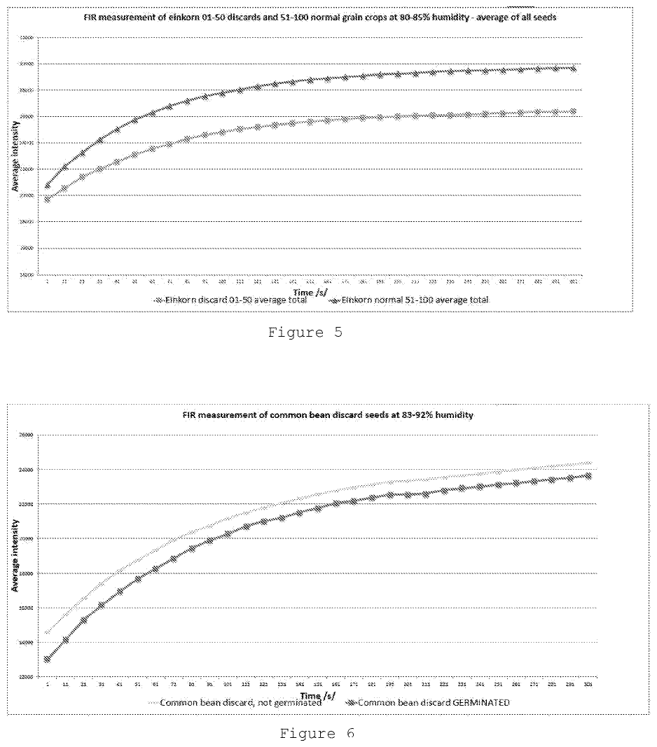

The seeds/grains of the common beans, maize and einkorn were germinated according to the standard for control. As can be seen from the thermal camera measurements of common beans (see FIG. 3), maize (see FIG. 4) and einkorn (see FIG. 5) seeds/grains, the “discard” (in our case this means non-viable seeds/grains) and in the case of “normal”, viable seeds/grains, the curves of average intensities show deviations depending on time. After standard germination of common beans, maize and einkorn seeds/grains, the results were compared with the results of thermal camera measurements. In the case of common beans, there were “discarded” seeds (4 of the 50 samples) that germinated, and there were “normal” seeds that did not germinate, or distorted sprouts that died (8 of the 50 samples). It is important to note that, according to the genebank protocol and Hungarian Standard (MSZ 1992), seeds that produce distorted sprouts are considered not germinated, as they are not expected to develop into plants. In the case of einkorn and maize, based on the germination results, the “discarded” seeds/grains really did not germinate, so they can really be considered as discards. Among the “normal” seeds/grains, however, there was an unexpectedly high proportion of non-germinating, moldy, and distorted grains. Due to the above, in the case of common beans, we further investigated whether the seeds that have germinated, but are considered “discards” based on the measurement, are separated from the non-germinated, truly “discarded” seeds based on the measurement results of the thermal camera tests. FIG. 6 clearly shows that, based on the thermal camera measurements, the non-germinated “discarded” seeds are separated from the germinated but considered “discarded” seeds, i.e. the measured average intensities differ. Furthermore, for all three species, we examined whether the non-germinated, but considered “normal” seeds are really different from the germinated “normal” seeds based on the measurement results of the thermal camera tests. The average intensity of the germinated seeds was separated from the non-germinated, but considered “normal” seeds based on thermal camera measurements. In particular, the average intensity values separated from each other in the 2nd uniform heating stage.

### Example 3: Separation of Maize Variety Groups

a) Prior to measurement, the maize seeds were dried in a drying chamber at 20° C. starting from a relative humidity of 20-22% with a continuous decrease in relative humidity (reaching and maintaining a relative humidity between 10-15%). With this method, we reached an einkorn moisture content of 6.1%.
b) The dried maize seeds according to step a) were stored for 24 hours at −18° C.±3° C. on the tray made of divided cardboard used for measurement (we placed 20 grains per tray, both genebank and hybrid maize species were tested), in a “no frost” freezer we cooled it.
c) The temperature of the walled space (measuring laboratory) was set to 16° C., the measured relative humidity was between 70-85 %.
d) As soon as possible, the tray containing the maize seeds cooled according to step b) was removed from the freezer and placed under the thermal camera and the thermal camera measurement was started. The measurement time was 10 minutes (there were no significant changes in the processes after that).
e) The thermal camera images of the maize seeds were processed by software.
f) Based on the results of the software processing, we drew a conclusion regarding the variety groups of maize seeds.

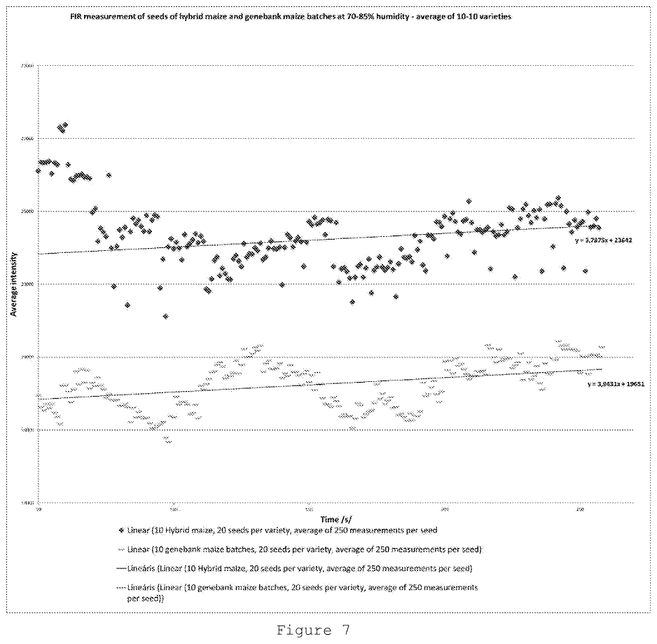

The measured values of the genebank maize batches are clearly distinct from the values measured with the thermal camera of the hybrid maize, there is a visible difference in the transient stages for the variety groups. Due to the sensitivity of the thermal camera, it is necessary to calibrate it, the alignment of the calibration sections is not indicated in the figure. The results are comparable based on the slope of the fitted curves and trend lines (see FIG. 7). The fitted linear trend line shows that in the case of hybrid maize and genebank maize batches, it is clearly an upward line (with a positive sign). However, the average intensity values and the intersection of the fitted straight line (trend line) with the y-axis are significantly different from each other, on the basis of which it is possible to identify unknown varieties of maize (hybrid or free-flowering) using the thermal camera measurement. In the case of bulbs and rhizomes, the plant parts should be cooled between 1-5° C., their warming can be examined similarly to that of the seeds.

The essential advantage of the system and method according to the present invention, compared to the state-of-the-art viability and/or varieties/variety group separation methods, is that it provides authentic results through thermal camera measurements in a completely non-destructive manner. In addition, with the measurement method according to the present invention, it is possible to obtain a lot of information about plant genetic resources that was not available until now. Thanks to the thermal camera of the system for testing the viability of plant genetic resources according to the present invention and/or identifying and separating their varieties/groups of varieties, it can be applied on all plant genetic resources, regardless of their shape, size, and tissue structure. The measurement times of the thermal camera measurements of the method according to the present invention are significantly shorter than those described in the state of the art, which is advantageous because the physiological processes necessary for germination are most likely not initiated. During the measurement, the plant genetic resources are not damaged, they can be measured repeatedly, and can still be used from a genebank and agricultural point of view (e.g. propagation).

1.-20. (canceled)

21. A system for the non-destructive identification and separation of viability for gene bank conservation and/or varieties/variety groups of plant genetic resources, wherein the system comprises at least the following:
a space enclosed by walls,
i) wherein temperature and humidity of the space is controllable;
ii) wherein the space contains one or more places suitable for measuring the plant genetic resources to be tested, and is suitable for placing trays for supporting the plant genetic resources;
a thermal camera positioned to face one or more trays suitable for storing the plant genetic resources to be measured;
a computer suitable for controlling the thermal camera and for storing and, where appropriate, processing the recordings made by the thermal camera;
a unit for drying the plant genetic resources to be tested;
a device for cooling and storing the plant genetic resources to be tested; and
one or more trays suitable for measuring the plant genetic resources to be tested.

22. A system for the non-destructive identification and separation of viability for gene bank conservation and/or varieties/variety groups of plant genetic resources, wherein the plant genetic resources are selected from the group consisting of: seeds and other plant parts.

23. The system of claim 22, wherein the plant genetic resource is a seed.

24. The system of claim 21, wherein the unit for drying is a drying chamber or other unit suitable for drying containing silica gel.

25. The system of claim 21, wherein the thermal camera is arranged in an enclosed measuring space together with a location for storing one or more trays for the plant genetic resources to be measured, and/or the thermal camera is mounted on a support stand.

26. The system of claim 21, wherein the spectral sensitivity of the thermal camera is between 7 and 14 μm.

27. The system of claim 21, wherein the one or more trays suitable for measuring the plant genetic resources have a split design.

28. The system of claim 21, wherein the material of the one or more trays suitable for measuring the plant genetic resources is paper, suitable plastic, or any natural or artificial insulating material; the material of the one or more trays suitable for measuring plant genetic resources is preferably cardboard.

29. A method for the non-destructive identification and separation of the viability for gene bank conservation and/or varieties/variety groups of plant genetic resources, wherein the method includes the following steps:
a) drying the plant genetic resources to be measured;
b) cooling said plant genetic resources after drying according to step a) to a temperature of 0° C. or below;
c) setting the temperature and humidity in the space for measurement, separated by walls;
d) placing the plant genetic resources to be measured, cooled according to step b) under a thermal camera and the measuring them with a thermal camera, where the measurement lasts no more than 2 hours;
e) software processing of thermal camera recordings of the named plant genetic resources; and
f) based on the result of the software processing obtained in step e), drawing a conclusion regarding the viability and/or variety/group of varieties of the measured plant genetic resources; where named plant genetic resources are placed on the one or more trays suitable for storing the plant genetic resources to be measured before step a), b) or c).

30. The method of claim 29, wherein the plant genetic resources are selected from the group comprising the following: seeds, other plant parts; the plant genetic resource is most preferably a seed.

31. The method of claim 29, wherein the temperature used in step a) during the drying of the plant genetic resources is between 10-25° C.

32. The method of claim 29, wherein in step a) during the drying of the plant genetic resources, the humidity is reduced so that it is finally set to a relative humidity between 10-15% in.

33. The method of claim 29, wherein the cooling of the plant genetic resources in step b) lasts at least 24 hours.

34. The method of claim 29, wherein during step b) the plant genetic resources to be measured are cooled to a temperature between 0° C. and −18° C.±3° C.

35. The method of claim 29, wherein for the cooling according to step b), the plant genetic resources are cooled in a sealed vessel and/or on a tray suitable for measurement.

36. The method of claim 29, wherein the temperature according to step c) has a temperature of 16° C.±4° C. and a relative humidity between 70-90%.

37. The method of claim 29, wherein in step d) the measurement time of the cooled plant genetic resources is up to 1 hour, preferably 30 minutes, most preferably 15 minutes.

38. The method of claim 29, wherein the method is carried out in accordance with a system for the non-destructive identification and separation of viability for gene bank conservation and/or varieties/variety groups of plant genetic resources, wherein the system comprises at least the following:
a space enclosed by walls,
i) wherein temperature and humidity of the space is controllable;
ii) wherein the space contains one or more places suitable for measuring the plant genetic resources to be tested, and is suitable for placing trays for supporting the plant genetic resources;
a thermal camera positioned to face one or more trays suitable for storing the plant genetic resources to be measured;
a computer suitable for controlling the thermal camera and for storing and, where appropriate, processing the recordings made by the thermal camera;
a unit for drying the plant genetic resources to be tested;
a device for cooling and storing the plant genetic resources to be tested; and
one or more trays suitable for measuring the plant genetic resources to be tested.

39. Use of the system for the non-destructive identification and separation of the viability for gene bank conservation and/or varieties/variety groups of plant genetic resources according to claim 21 for the non-destructive identification and separation of the viability and/or varieties/variety groups of plant genetic resources.

40. The use of the system for the non-destructive identification and separation of the viability for gene bank conservation and/or varieties/groups of plant genetic resources according to claim 21 for the non-destructive identification and separation of the viability for gene bank conservation and/or varieties/variety groups of plant genetic resources, wherein the plant genetic resources are selected from the group comprising the following: seeds other plant parts and the tested plant genetic resources remain suitable for further genebank conservation; the plant genetic resource is most preferably a seed.
